(19) United States
(12) Patent Application Publication
(10) Pub. No.: US 2015/0289844 A1
(43) Pub. Date: Oct. 15, 2015

(54) DIAGNOSTIC ULTRASOUND IMAGING DEVICE

(71) Applicant: Konica Minolta, Inc., Tokyo (JP)

(72) Inventor: Yukiko OKAMOTO, Yokohama-shi (JP)

(21) Appl. No.: 14/672,915

(22) Filed: Mar. 30, 2015

(30) Foreign Application Priority Data
Apr. 9, 2014 (JP) 2014-080056

(51) Int. Cl. A61B 8/00; A61B 8/08; A61B 8/14

(52) U.S. Cl. CPC A61B 8/469; A61B 8/14; A61B 8/465; A61B 8/463; A61B 8/5207

(57) ABSTRACT

A diagnostic ultrasound imaging device is connectable to an ultrasound probe which has a piezoelectric element. The diagnostic ultrasound imaging device includes an image generation unit, a display unit, a touch panel and a control unit. The image generation unit generates an ultrasound image on the basis of a reception signal obtained from received reflected ultrasound from a subject. The display unit displays the generated ultrasound image on a display screen. The touch panel detects an operation on the display screen. When the touch panel detects on the displayed ultrasound image an end of a cursor movement operation to move a cursor, the control unit displays near the moved cursor an operation button to activate a predetermined function.

### 1. FIELD OF THE INVENTION

The present invention relates to a diagnostic ultrasound imaging device.

### 2. DESCRIPTION OF THE RELATED ART

There has been known a diagnostic ultrasound imaging apparatus which transmits ultrasound with an ultrasound probe abutting the surface of a subject such as a living body, receives with the ultrasound probe reflected ultrasound (echo) of the ultrasound reflected by the subject, and generates ultrasound images (tomograms) of the inside of the subject according to the received ultrasound.

The diagnostic ultrasound imaging apparatus is provided with a measurement function to measure the distance, area, volume and so forth about a target part in a subject.

In the measurement function, measurements are performed, for example, on the basis of position coordinates of multiple points on an ultrasound image, the multiple points being specified by an operator. For example, when the length of a target part is measured, the start point and the end point thereof are specified on an ultrasound image, position coordinates of the specified two points are obtained, the distance (length) between the two points are measured, and the measurement result is displayed on a monitor or the like.

The position coordinates used for the measurement are specified, for example, by displaying slide calipers (simply called “calipers”) on the ultrasound image, and with a control panel (operation button(s)) provided in the diagnostic ultrasound imaging apparatus, moving the calipers to the target part on a screen and performing a setting operation at a predetermined position. (Refer to, for example, Japanese Patent Application Laid-Open Publication No. 2009-82365.) There is a diagnostic ultrasound imaging apparatus in which such position coordinates are specified, not with a control panel but with a touch panel provided on a monitor, by moving a measurement cursor and performing a setting operation.

In this type of diagnostic ultrasound imaging apparatus, which performs measurements with a touch panel, an operation button (Set button) to perform a setting operation to set the position of a measurement cursor after the measurement cursor being moved is displayed at a fixed position on a screen. In diagnostic ultrasound imaging, an operator does the work holding an ultrasound probe with one hand, and accordingly needs to perform operations on the touch panel with the other one hand. Therefore, after moving the measurement cursor to a position, the operator needs to move his/her finger from the position of the measurement cursor to the fixed position, where the Set button is displayed, so as to set the position of the measurement cursor. These operations are troublesome.

### BRIEF SUMMARY OF THE INVENTION

Objects of the present invention include increasing usability of a diagnostic ultrasound imaging device provided with a touch panel.

In order to achieve at least one of the objects, according to an aspect of the present invention, there is provided a diagnostic ultrasound imaging device connectable to an ultrasound probe having a piezoelectric element, the diagnostic ultrasound imaging device including: an image generation unit which generates an ultrasound image on the basis of a reception signal obtained from received reflected ultrasound from a subject; a display unit which displays the generated ultrasound image on a display screen; a touch panel which detects an operation on the display screen; and a control unit which, when the touch panel detects on the displayed ultrasound image an end of a cursor movement operation to move a cursor, displays near the moved cursor an operation button to activate a predetermined function.

Preferably, the control unit displays the operation button a predetermined first reference distance or more and a predetermined second reference distance or less away from the moved cursor, the second reference distance being greater than the first reference distance.

Preferably, the control unit displays the operation button at a position which does not overlap a movement direction of the cursor.

Preferably, when the touch panel detects not a press of the operation button but an end of the cursor movement operation after the control unit displays the operation button, the control unit moves the operation button to near the again-moved cursor.

Preferably, the cursor movement operation is an operation to slide the cursor to a destination while holding down the cursor and release the cursor at the destination.

Preferably, the cursor movement operation is an operation to press a position of a destination for the cursor and stop pressing the position.

Preferably, the operation button is a button to set a position of the cursor at a position of the moved cursor.

Preferably, the operation button is a button to select an item of a measurement function.

### BRIEF DESCRIPTION OF THE SEVERAL VIEWS OF THE DRAWING

The present invention is fully understood from the detailed description given hereinafter and the accompanying drawings, which are given byway of illustration only and thus are not intended to limit the present invention, wherein:

FIG. 1 shows the external appearance of a diagnostic ultrasound imaging apparatus;

FIG. 2 is a block diagram schematically showing the configuration of the diagnostic ultrasound imaging apparatus;

FIG. 3 shows operations on an examination screen to proceed to a measurement mode;

FIG. 4 is a flowchart of a Set button display control process performed by a control unit shown in FIG. 2;

FIG. 5A shows a display example in an image display region on the examination screen shown when an operator presses a distance measurement button;

FIG. 5B shows an example of a movement operation on a start point cursor performed by the operator;

FIG. 5C shows a display example in the image display region on the examination screen shown when the operator moves the start point cursor to a desired position;

FIG. 5D shows a display example in the image display region on the examination screen shown when the operator presses a Set button displayed near the start point cursor;

FIG. 5E shows a display example in the image display region on the examination screen shown when the operator performs the movement operation on an end point cursor to move the end point cursor to a desired position; and

FIG. 5F shows a display example in the image display region on the examination screen shown when the operator presses the Set button displayed near the end point cursor.

### DETAILED DESCRIPTION OF THE INVENTION

Hereinafter, a diagnostic ultrasound imaging apparatus according to an embodiment of the present invention is described with reference to the drawings. However, the scope of the present invention is not limited to the illustrated examples.

### Configuration of Diagnostic Ultrasound Imaging Apparatus

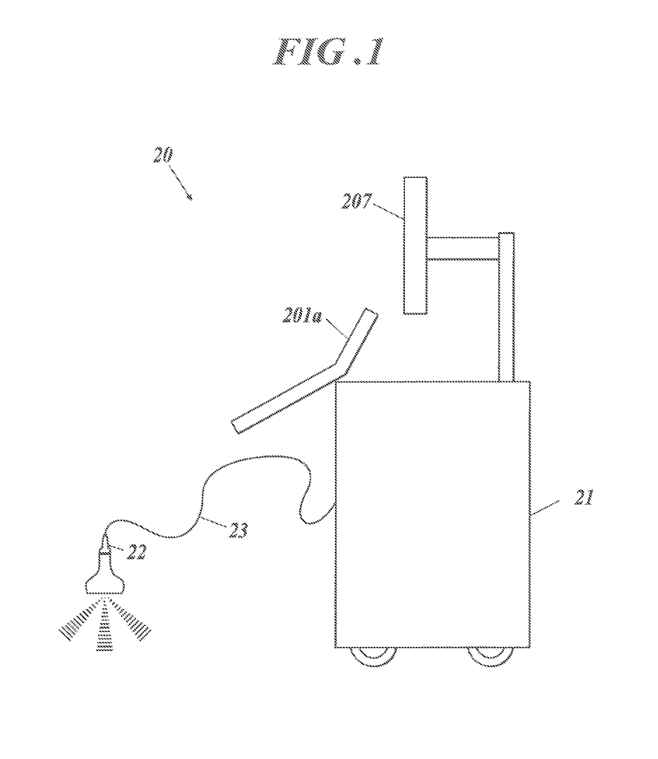

A diagnostic ultrasound imaging apparatus 20 of the embodiment includes, as shown in FIG. 1, a main body 21 (a diagnostic ultrasound imaging device) and an ultrasound probe 22 connectable thereto. The ultrasound probe 22 transmits ultrasound (transmission ultrasound) to a not-shown subject such as a living body and receives reflected waves (reflected ultrasound or echo) generated by the ultrasound being reflected by the subject. The main body 21 is connected to the ultrasound probe 22 through a cable 23 and transmits drive signals composed of electric signals to the ultrasound probe 22 so as to make the ultrasound probe 22 transmit the transmission ultrasound to a subject and images the internal state of the subject as ultrasound images on the basis of reception signals composed of electric signals which the ultrasound probe 22 generates according to the reflected ultrasound received from the inside of the subject. The connection between the main body 21 and the ultrasound probe 22 is not limited to the cable connection through the cable 23 and hence may be wireless connection through radio waves, infrared rays, light or the like.

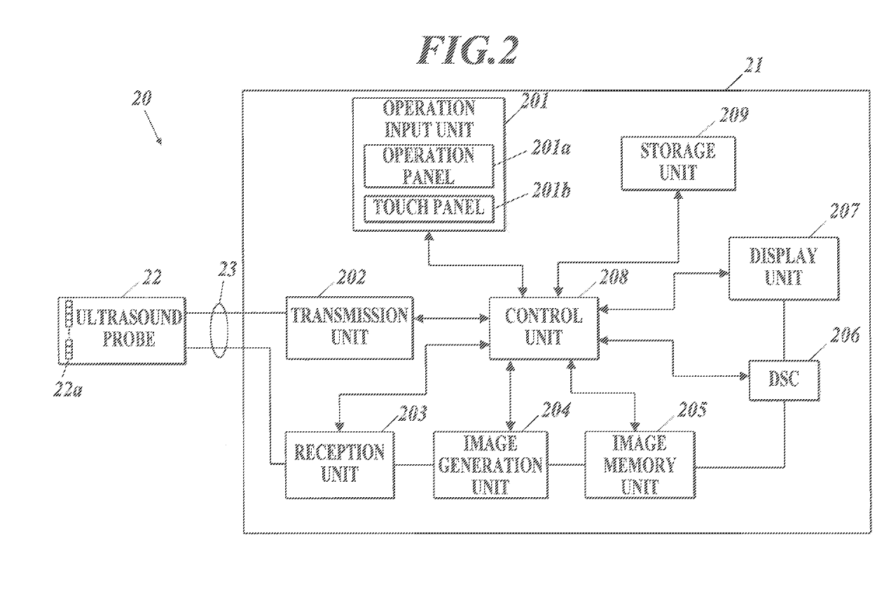

The ultrasound probe 22 includes transducers 22a (see FIG. 2) constituted of piezoelectric elements, and these transducers 22a are arranged in a one-dimensional array in an orientation direction. In the embodiment, the ultrasound probe 22 having 192 transducers 22a is used as an example. The transducers 22a may be arranged in a two-dimensional array. The number of transducers 22a can be appropriately set. As the scanning mode of the ultrasound probe 22, either of an electronic scanning mode and a mechanical scanning mode may be adopted, and also any of a linear scanning mode, a sector scanning mode and a convex scanning mode may be adopted.

The main body 21 includes, for example, as shown in FIG. 2, an operation input unit 201, a transmission unit 202, a reception unit 203, an image generation unit 204, an image memory unit 205, a DSC (Digital Scan Converter) 206, a display unit 207, a control unit 208 and a storage unit 209.

The operation input unit 201 includes an operation panel 201a and a touch panel 201b. The operation panel 201a includes: for example, various switches and buttons, a trackball, a mouse and a keyboard to input commands to start diagnosis (diagnostic ultrasound imaging), to input data such as personal information about subjects, to perform freeze operations and so forth, and outputs operation signals to the control unit 208. The touch panel 201b is integrated with a display of the display unit 207, and detects operations performed with a finger or the like on a display screen of the display unit 207 and outputs coordinate information about the operated positions to the control unit 208.

The transmission unit 202 is a circuit to supply the drive signals, which are composed of electric signals, to the ultrasound probe 22 through the cable 23 under the control of the control unit 208 so as to make the ultrasound probe 22 generate the transmission ultrasound. The transmission unit 202 includes, for example, a clock generation circuit, a delay circuit and a pulse generation circuit. The clock generation circuit is a circuit to generate clock signals which determine transmission timings of the drive signals and the transmission frequency thereof. The delay circuit is a circuit to individually set delay times with respect to respective paths for the transducers 22a, the delay times by which the transmission timings of the drive signals are delayed, and to delay transmission of the drive signals by the set delay times so as to focus transmission beams composed of the transmission ultrasound. The pulse generation circuit is a circuit to generate pulse signals as the drive signals at predetermined intervals.

Thus-configured transmission unit 202 successively changes, under the control of the control unit 208, the transducers 22a each time ultrasound is transmitted/received so as to supply the drive signals to a predetermined number of transducers 22a, and supplies the drive signals to the transducers 22a selected to output the transmission ultrasound to a subject, whereby a scan is performed.

The reception unit 203 is a circuit to receive the reception signals, which are composed of electric signals, from the ultrasound probe 22 through the cable 23 under the control of the control unit 208. The reception unit 203 includes, for example, an amplifier, an A/D converter circuit and a phasing addition circuit. The amplifier is a circuit to amplify the reception signals at a predetermined amplification factor(s) present with respect to the respective paths for the transducers 22a. The A/D converter circuit is a circuit to perform analog-to-digital (A/D) conversion on the amplified reception signals. The phasing addition circuit is a circuit to give the delay times, which are individually set with respect to the respective paths for the transducers 22a, to the A/D conversion-performed reception signals so as to phase the signals and add up these signals (phasing addition), thereby generating sound ray data.

The image generation unit 204 performs envelope detection, logarithmic amplification or the like on the sound ray data received from the reception unit 203 and performs gain control or the like so as to perform brightness conversion, thereby generating B-mode images. That is, the B-mode images express strengths of the reception signals by brightness. The B-mode images generated by the image generation unit 204 are sent to the image memory unit 205. The image generation unit 204 may be capable of generating M-mode images, Doppler images and the like besides the B-mode images.

The image memory unit 205 is constituted of a semiconductor memory such as a DRAM (Dynamic Random Access Memory) and stores therein the B-mode images frame-by-frame, the B-mode images being output from the image generation unit 204. The frame-by-frame B-mode images are referred to as ultrasound images or frame images. The image memory unit 205 is constituted of a mass memory to hold image data of ultrasound images of about 10 seconds. For example, the image memory unit 205 holds ultrasound images of the most recent 10 seconds by FIFO (First-In First-Out). The ultrasound images are output from the image memory unit 205 to the DSC 206 frame-by-frame at predetermined time intervals under the control of the control unit 208.

In the embodiment, the B-mode images are generated as ultrasound images. Alternatively, the M-mode images, the Doppler images or the like may be generated as ultrasound images.

The DSC 206 converts the ultrasound images received from the image memory unit 205 into image signals in accordance with a television signal scanning mode and outputs the image signals to the display unit 207.

As the display unit 207, a display device such as an LCD (Liquid Crystal Display), a CRT (Cathode-Ray Tube) display, an organic EL (Electronic Luminescence) display, an inorganic EL display or a plasma display can be used. The display unit 207 displays ultrasound images on the display screen in response to the image signals output from the DSC 206.

The control unit 208 includes, for example, a CPU (Central Processing Unit), a ROM (Read Only Memory) and a RAM (Random Access Memory), and reads various process programs including a system program stored in the ROM, opens the read programs in the RAM and performs centralized control of actions of the components of the diagnostic ultrasound imaging apparatus 20 in accordance with the opened programs.

The ROM is constituted of, for example, a nonvolatile memory such as a semiconductor memory and stores therein, for example, the system program for the diagnostic ultrasound imaging apparatus 20, the various process programs executable on the system program and various data. These programs are stored in a program code readable by a computer, and the CPU acts in accordance with the program code.

The RAM forms a work area where the various programs to be executed by the CPU and data relevant to the programs are temporarily stored.

The storage unit 209 is constituted of, for example, an HDD (Hard Disk Drive) or a nonvolatile semiconductor memory. The storage unit 209 stores therein, for example, various types of setting information of the diagnostic ultrasound imaging apparatus 20 and ultrasound images for which commands to save are made.

### [Action in Measurement Mode]

The diagnostic ultrasound imaging apparatus 20 is provided with a measurement mode to perform various measurements about a target part in a subject (for example, a structure or a lesion part in a living body) on the basis of generated ultrasound images. In the measurement mode, for example, the following can be measured; the distance between two points, the trace length of a traced shape, the diameter, circumference and area of a circle or an ellipse, angle, time and volume.

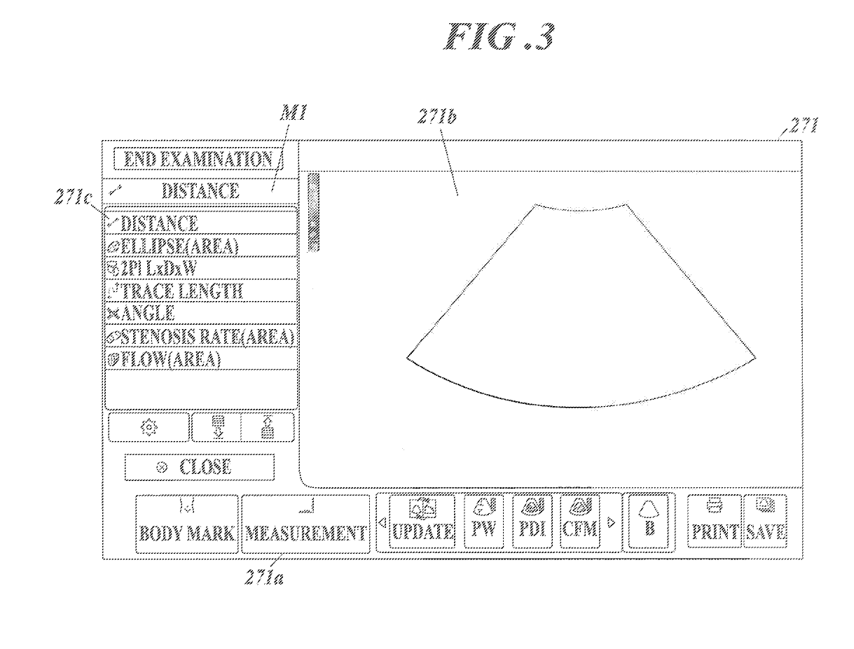

For example, as shown in FIG. 3, when a measurement button 271a is pressed in a state in which an examination screen 271 to display captured ultrasound images is displayed on the display unit 207, the measurement mode starts and a measurement menu M1 (a button to select an item of a measurement function) is displayed on the examination screen 271. When a desired item button of the measurement menu M1 is pressed (selected) in a state in which an ultrasound image of a measurement target is displayed in an image display region 271b, a measurement cursor is displayed on the ultrasound image and a measurement corresponding to the selected item is performed in response to user operations on the measurement cursor and so forth. The measurement cursor is a mark to indicate the current position of a point, an axis or the like necessary for a measurement, and examples thereof include a start point cursor C1 and an end point cursor C2 described below as well as the calipers in the conventional art.

The ultrasound image of a measurement target can be displayed, for example, by pressing the freeze button on the operation panel 201a when a target part which is the measurement target is shown in an ultrasound image which is obtained by pressing the ultrasound probe 22 against a subject.

In the measurement mode, a measurement is mainly performed on the basis of position coordinates on an ultrasound image, the position coordinates being specified by an operator. The position coordinates used for a measurement are specified, in the case where the touch panel 201 is used, by an operator performing a movement operation on the measurement cursor displayed on an ultrasound image of a measurement target to move the measurement cursor to a desired position and performing a setting operation, such as pressing a Set button, to set the position of the measurement cursor at the position of the moved measurement cursor . However, in the case where the Set button is displayed at a fixed position on the screen as with the conventional art, when the operator performs the above-described operations with one hand, the operator needs, after moving the measurement cursor to the desired position, to move his/her finger from the position of the measurement cursor to the fixed position (outside the image display region), where the Set button is displayed, so as to set the position of the measurement cursor thereat. These operations are troublesome.

Hence, in the embodiment, the control unit 208 performs a Set button display control process after displaying the measurement cursor on an ultrasound image displayed in the image display region 271b, thereby, when the touch panel 201b detects the end of the movement operation on the measurement cursor, displaying the Set button near the moved measurement cursor. Consequently, the setting operation to set the position coordinates can be performed without moving a finger almost at all.

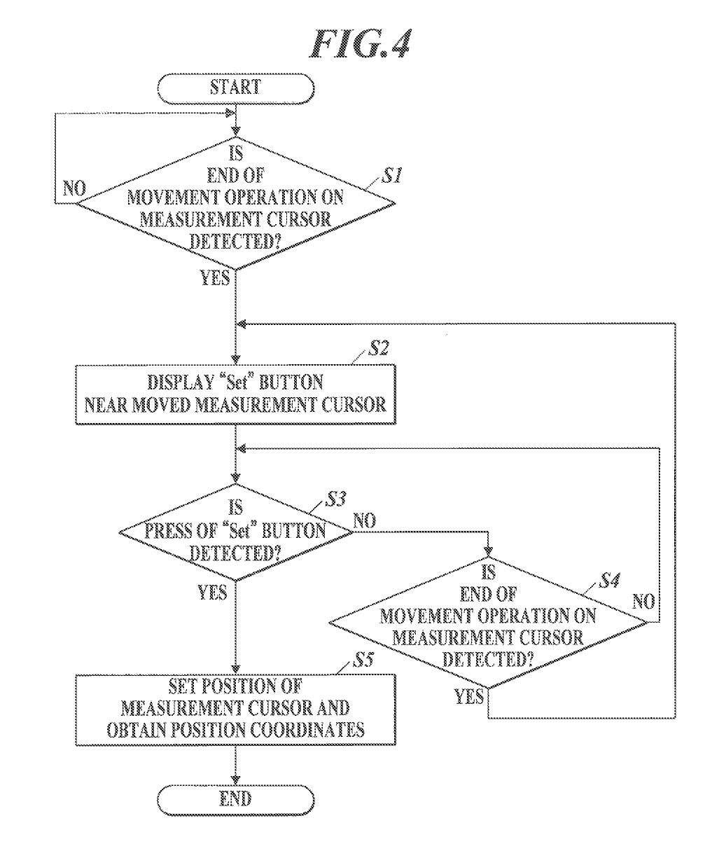

Hereinafter, the Set button display control process performed by the control unit 208 is described with reference to FIG. 4.

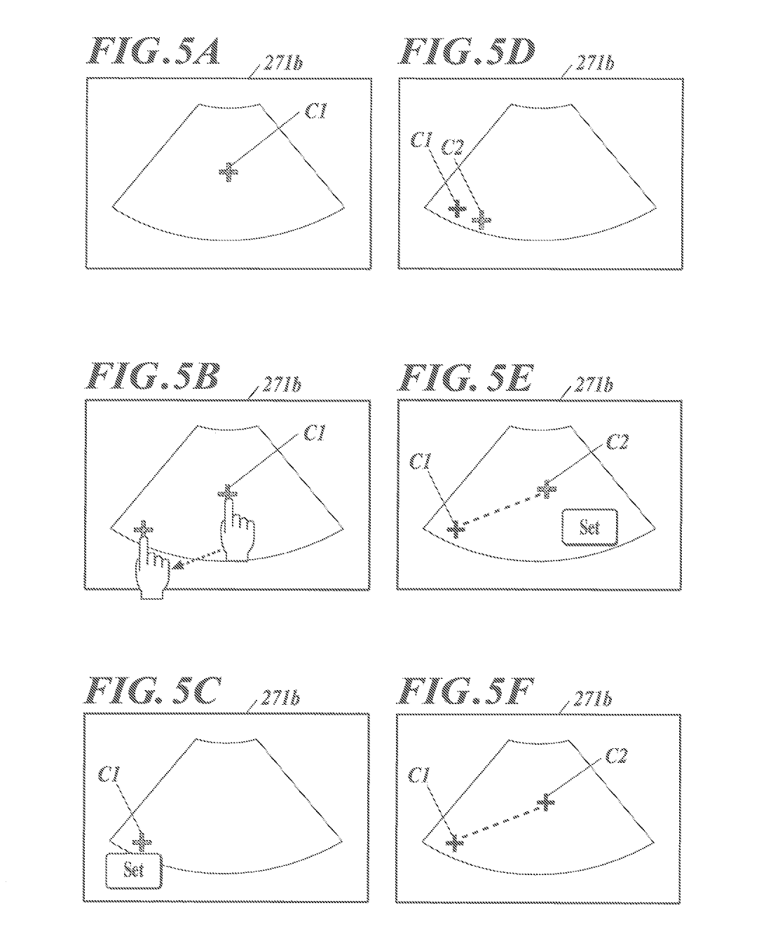

First, the control unit 208 waits until the touch panel 201b detects an end of the movement operation on the measurement cursor displayed on an ultrasound image on the examination screen 271 (Step S1). Examples of the method for the movement operation on the measurement cursor include: (1) a method of pressing the measurement cursor with a finger or the like, sliding the measurement cursor to a destination while holding down the measurement cursor with the finger, and releasing the finger from the measurement cursor (i.e., releasing the measurement cursor) at the destination (i.e., “drag”, see FIG. 5B); and (2) a method of directly pressing the position of a destination for the measurement cursor with a finger or the like, and releasing the finger from the position (i.e., stopping pressing the position) of the destination (i.e., “click”). In the above two methods, the timing when the finger is released is detected as the end of the movement operation. Alternatively, the timing when a predetermined time elapses (without finger release) from the time that the operation to press and slide the measurement cursor stops or the timing when a predetermined time elapses (without finger release) from the time that the position other than the measurement cursor is pressed may be detected as the end of the movement operation.

When determining that the touch panel 201b detects an end of the movement operation on the measurement cursor (Step S1; YES), the control unit 208 displays the Set button near the moved measurement cursor (Step S2).

The “near the measurement cursor” is, for example, a position within a predetermined reference distance (second reference distance) from the center of the displayed measurement cursor. In order to prevent false detection, this position is preferably in an area where a finger does not overlap the Set button when a finger which is normal in size (thickness) is placed on the center of the measurement cursor, namely, this position is preferably a predetermined reference distance (first reference distance) or more away from the center of the measurement cursor. Note that “the first reference distance<the second reference distance”. That is, the Set button is preferably displayed the first reference distance or more and the second reference distance or less away from the measurement cursor. Further, the Set button is preferably displayed at a position which does not overlap a movement direction of the measurement cursor in consideration of readjustment of the position of the measurement cursor.

Next, the control unit 208 determines whether or not the touch panel 201b detects a press of the Set button (Step S3).

When determining that the touch panel 201b does not detect a press of the Set button (Step S3; NO), the control unit 208 determines whether or not the touch panel 201b detects an end of the movement operation on the measurement cursor (Step S4). When determining that the touch panel 201b detects an end of the movement operation on the measurement cursor (Step S4; YES), the control unit 208 returns to Step S2 so as to display the Set button near the again-moved measurement cursor. That is, when the measurement cursor is moved after the Set button is displayed, the Set button is moved in response to the movement of the measurement cursor.

On the other hand, when determining that the touch panel 201b does not detect an end of the movement operation on the measurement cursor (Step S4; NO), the control unit 208 returns to Step S3.

When determining that the touch panel 201b detects a press of the Set button (Step S3; YES), the control unit 208 sets the position of the measurement cursor, obtains position coordinates thereof and changes the color of the measurement cursor to a set color which indicates that the position of the measurement cursor is set (Step S5), and then ends the Set button display control process.

Here, display examples of the user interface the display of which is controlled by the above-described Set button display control process are described with reference to FIGS. 5A to 5F. Hereinafter, display examples of the user interface in measuring the distance between two points are described. In FIGS. 5A to 5F, with respect to each of the start point cursor C1 and the end point cursor C2, gray is an active color which indicates that the cursor is active, and black is the set color which indicates that the position of the cursor is set.

When an operator presses a distance measurement button 271c of the measurement menu M1, as shown in FIG. 5A, the start point cursor C1 is displayed on an ultrasound image displayed in the image display region 271b of the examination screen 271.

When, as shown in FIG. 5B, the operator performs the movement operation on the start point cursor C1 to move the start point cursor C1 to a desired position, as shown in FIG. 5C, the Set button is displayed near the moved start point cursor C1.

When the operator performs the movement operation on the start point cursor C1 again without pressing the Set button, the Set button is moved to near the again-moved start point cursor C1.

When the operator presses the Set button, as shown in FIG. 5D, the position of the start point cursor C1 is set (the color of the start point cursor C1 is changed to the set color), and the end point cursor C2 is displayed near the start point cursor C1.

When the operator performs the movement operation on the end point cursor C2 to move the end point cursor C2 to a desired position, as shown in FIG. 5E, the Set button is displayed near the moved end point cursor C2.

When the operator performs the movement operation on the end point cursor C2 again without pressing the Set button, the Set button is moved to near the again-moved end point cursor C2.

When the operator presses the Set button, as shown in FIG. 5F, the position of the end point cursor C2 is set (the color of the end point cursor C2 is changed to the set color).

The control unit 208 measures the distance between the start point cursor C1 and the end point cursor C2 on the basis of the set position coordinates of the start point cursor C1 and the set position coordinates of the end point cursor C2 and displays the measurement result on the examination screen 271.

As described above, according to the diagnostic ultrasound imaging apparatus 20, when the touch panel 201b detects an end of the movement operation on the measurement cursor on an ultrasound image displayed on the display screen of the display unit 207, the control unit 208 displays near the moved measurement cursor the Set button to set the position of the measurement cursor at the position of the moved measurement cursor.

Consequently, the movement amount of a finger to perform the setting operation to set the position of the measurement cursor, the setting operation being often performed, can be substantially reduced, and hence while doing diagnostic ultrasound imaging holding a probe with one hand, an operator can easily operate the measurement cursor with the other one hand. Therefore, usability of the diagnostic ultrasound imaging apparatus 20, which is provided with the touch panel 201b, in measurements can be increased.

Further, the Set button is displayed the predetermined first reference distance or more and the predetermined second reference distance or less away from the measurement cursor so that, for example, when a finger is placed on the moved measurement cursor, the finger does not overlap the Set button. Consequently, false detection can be prevented while the usability can be increased.

Further, the Set button is displayed at a position which does not overlap the movement direction of the measurement cursor. Consequently, it can be prevented from happening that the Set button becomes an obstacle to adjustment of the position of the once-moved measurement cursor.

Further, when the touch panel 201b detects not a press of the Set button but an end of the movement operation on the measurement cursor after the control unit 208 displays the Set button, the control unit 208 moves the Set button to near the again-moved measurement cursor. Consequently, even when the position of the measurement cursor is adjusted, the setting operation to set the position of the measurement cursor can be easily performed.

The above-described embodiment of the present invention is an example of the diagnostic ultrasound imaging apparatus (and the diagnostic ultrasound imaging device) of the present invention, and hence the present invention is not limited thereto.

For example, in the embodiment, as an example of the user interface to which the Set button display control process is applied, the user interface displayed in measuring the distance between two points is described. However, this is not a limitation but an example. Hence, the Set button display control process performed by the present invention is applicable to other measurements. Further, the present invention is not limited to the measurement mode and hence is applicable to any mode or the like in which positions are specified with a cursor(s) on a touch panel.

Further, in the embodiment, the Set button is displayed and its function is described. However, the operation button displayed near the cursor is not limited to the Set button.

For example, the measurement menu M1 may be displayed near the cursor. In the above descriptions with reference to FIG. 3, the measurement menu M1 is displayed when the measurement button 271a is pressed. Alternatively, the control unit 208 may immediately display the measurement cursor when the measurement button 271a is pressed, and display the measurement menu M1 near the cursor when the touch panel 201b detects that an operator moves the cursor. In this case, when one of the item buttons for items of the measurement function, for example, the distance measurement button 271c, of the measurement menu M1 displayed near the cursor is pressed, the distance measurement mode starts.

This action simplifies the operation procedure for measurements and further increases the usability.

The present invention is not limited to these examples and hence intended to display, when the cursor is displayed, any operation button to activate a function which should be activated next.

Further, in the embodiment, a ROM, a hard disk, a semiconductor nonvolatile memory or the like is used as a computer readable storage medium to store the programs of the present invention. However, this is not a limitation, and hence a portable storage medium such as a CD-ROM is also usable as the computer readable storage medium. Further, carrier waves are usable as a medium to provide data of the programs of the present invention via a communication line.

Besides, the detailed configurations and actions of the functional units of the diagnostic ultrasound imaging apparatus can also be appropriately modified.

This application is based upon and claims the benefit of priority under 35 U.S.C. 119 to Japanese Patent Application No. 2014-080056 filed Apr. 9, 2014, the entire disclosure of which, including the specification, claims, drawings and abstract, is incorporated herein by reference in its entirety.

What is claimed is:

1. A diagnostic ultrasound imaging device connectable to an ultrasound probe having a piezoelectric element, the diagnostic ultrasound imaging device comprising:
an image generation unit which generates an ultrasound image on the basis of a reception signal obtained from received reflected ultrasound from a subject;
a display unit which displays the generated ultrasound image on a display screen;
a touch panel which detects an operation on the display screen; and
a control unit which, when the touch panel detects on the displayed ultrasound image an end of a cursor movement operation to move a cursor, displays near the moved cursor an operation button to activate a predetermined function.

2. The diagnostic ultrasound imaging device according to claim 1, wherein the control unit displays the operation button a predetermined first reference distance or more and a predetermined second reference distance or less away from the moved cursor, the second reference distance being greater than the first reference distance.

3. The diagnostic ultrasound imaging device according to claim 1, wherein the control unit displays the operation button at a position which does not overlap a movement direction of the cursor.

4. The diagnostic ultrasound imaging device according to claim 1, wherein when the touch panel detects not a press of the operation button but an end of the cursor movement operation after the control unit displays the operation button, the control unit moves the operation button to near the again-moved cursor.

5. The diagnostic ultrasound imaging device according to claim 1, wherein the cursor movement operation is an operation to slide the cursor to a destination while holding down the cursor and release the cursor at the destination.

6. The diagnostic ultrasound imaging device according to claim 1, wherein the cursor movement operation is an operation to press a position of a destination for the cursor and stop pressing the position.

7. The diagnostic ultrasound imaging device according to claim 1, wherein the operation button is a button to set a position of the cursor at a position of the moved cursor.

8. The diagnostic ultrasound imaging device according to claim 1, wherein the operation button is a button to select an item of a measurement function.
